(19) United States
(12) Patent Application Publication
(10) Pub. No.: US 2010/0010338 A1
(43) Pub. Date: Jan. 14, 2010

(54) Implantable Medical Device Orientation Detection Utilizing an External Magnet and a 3D Accelerometer Sensor

(76) Inventors: Peter van Dam, Doesburg (NL); Patrick Scholten, SK Lettele (NL)

Correspondence Address: Medtronic, Inc., 710 Medtronic Parkway, Mail Stop LC340, Minneapolis, MN, 55432, US

(21) Appl. No.: 12/242,798

(22) Filed: Sep. 30, 2008

Related U.S. Application Data
(60) Provisional application No. 61/079,066, filed on Jul. 8, 2008.

(51) Int. Cl. A61B 5/05; A61N 1/00

(52) U.S. Cl. 600/424; 607/60

(57) ABSTRACT

A method and device for detecting the implanted orientation of an implantable medical device (IMD) in a patient. IMD includes an accelerometer for measuring acceleration signals in three orthogonal directional axes. A y-axis orientation of IMD is determined from the measured accelerometer signals using a gravitational force analysis. IMD includes a magnetic sensor that senses a varying magnetic field exerted on the magnetic sensor from an external magnet moved along a medial-lateral direction with respect to IMD. The z-axis orientation of IMD is determined from the location of the external magnet where the magnetic field exerted on the magnetic sensor is greatest. Based on a known relationship between the accelerometer and magnetic sensor, an orthogonal transformation calculation is performed on the y-axis and z-axis orientations to yield the x-axis orientation. The implanted orientation of IMD with respect to the patient is thus known and used to compensate accelerometer measurements.

### RELATED APPLICATION

This application claims the benefit of and priority to U.S. Provisional Application Ser. No. 61/079,066, filed Jul. 8, 2008, entitled, “Implantable Medical Device Orientation Detection Utilizing an External Magnet and a 3D Accelerometer Sensor,” the contents of which are incorporated by reference herein in its entirety.

### TECHNICAL FIELD

This disclosure relates generally to medical devices and more particularly to a method and device for the detection of the implanted orientation of an implantable medical device in a patient.

### BACKGROUND

A pulse generator is one of many medical devices that are implantable in a patient and provide a therapy that is dependent on the current activity level of the patient. For example, a pacemaker is a widely used medical device that includes a pulse generator for providing stimulus to cardiac tissue. The amount of stimulus provided corresponds to the activity level of the patient. A patient that is sleeping requires lower stimuli than a person that is active and in motion. One method for determining the activity level of the patient is to use an accelerometer.

An accelerometer measures changes in a patient's physical activity. The physical changes are detected by the accelerometer and algorithmically interpreted by circuitry within the pulse generator to produce a modified therapy that is correct for the current activity level or, for instance, to manage a shock because the patient is determined to be lying on the ground and a ventricular tachycardia (VT) is detected. The accelerometer is placed within the implantable medical device. One type that has been successfully implemented in a pulse generator is a single axis accelerometer that measures both dynamic and static acceleration (e.g. gravity) in a single direction. Measurement in all three dimensions is achieved by using three single axis accelerometers respectively mounted to detect in the x, y, and z axis (a “3D accelerometer”). In order to accurately detect physical changes in the physical activity of a patient, the output signals from the 3D accelerometer sensor in a subcutaneous or implantable medical device must be compensated for the implanted orientation of the device in the patient.

### SUMMARY

A method and device are provided for detecting the implanted orientation of an implantable medical device in a patient and then optimizing or compensating for such implanted orientation. The method and device determine the orientation of the implantable medical device with respect to the patient utilizing information received from accelerometer and magnetic sensors contained within the implantable medical device. In one or more embodiments, the device includes a three-dimensional (“3D”) 3D accelerometer sensor configured for measuring acceleration signals in three orthogonal directional axes comprising an x-axis, z-axis and y-axis. A processor or controller is connected to the 3D accelerometer for receiving the measured acceleration signals and determining an orientation of the implantable medical device with respect to gravity from the measured accelerometer signals (i.e., a y-axis orientation). In some embodiments, the y-axis orientation of the implantable medical device is determined when it is known that the patient in which the implantable medical device is implanted is in an upright position or posture. In some embodiments, the controller is configured to sense when the patient is walking by monitoring the accelerometer signals received from the 3D accelerometer to detect certain low frequency periodic signals that can be used to identify that the patient is walking, wherein the y-axis orientation is determined when it is known that the patient is in an upright walking position or performing similar detectable physical activity associated with an upright patient position. In some circumstances, only the orientation of the y-axis is necessary, such as for fall detection techniques. In other circumstances, such as when required for posture classification or identification, the exact orientation of the other x-axis and z-axis orientations with respect to the inclination of the device are required.

In one or more embodiments, the implantable medical device includes a magnetic sensor configured to sense a magnetic field exerted on the magnetic sensor from a magnetic field source, where the magnetic sensor and the accelerometer have a fixed relationship with one another. In some embodiments, the magnetic field source may include an external magnet that is movable with respect to the magnetic sensor so that the position of the external magnet is moved substantially along a direction of a lateral-medial axis of the patient or device (i.e., on the anterior side of the patient, the external magnet is moved between left and right sides of the patient along a direction that is substantially consistent with the lateral-medial axis of the patient). In some embodiments, the same respective movement is achieved by retaining the external magnet in a stationary position while the patient moves with respect to the external magnet. As the external magnet moves with respect to the magnetic sensor, the strength of the magnetic field exerted on the magnetic sensor will vary. The controller is connected to the magnetic sensor for receiving magnetic field signals relating the varying magnetic field imparted on the magnetic sensor. In one or more embodiments, the controller is configured to determine the maximum value of the magnetic field signals it receives and to identify the corresponding location of the external magnet associated with the maximum value of the magnetic field. The z-axis orientation of the device can be determined based on the location at which the magnetic field has a maximum value. In some embodiments, rather than using a moving magnetic field source, a magnetic field source having a stationary and known position may be used, where the strength of the magnetic field exerted on the magnetic sensor from the stationary magnetic field source can be measured and used to calculate the z-axis orientation of the device.

In one or more embodiments, the orientation between the 3D accelerometer sensor and the magnetic sensor within the implantable medical device is known and fixed. Thus, the controller is configured for determining an x-axis orientation of the device from an orthogonal transformation calculation using the previously determined y-axis orientation from the accelerometer signals and the z-axis orientation from the magnetic field signals. In this manner, once the x-axis, y-axis and z-axis orientations of the device are known, the implanted orientation of the device within the patient can be determined and the accelerometer signals received from the 3D accelerometer sensor can be compensated for by the controller. In this manner, the 3D accelerometer sensor is essentially calibrated such that its output signals can be adjusted to account for the implanted orientation or inclination of the implantable medical device in the patient. In some embodiments, the calibration procedures can be repeated at various points in time to re-calibrate the implanted orientation of the implantable medical device to account for movement of the implantable medical device within the patient.

### DRAWINGS

The above-mentioned features and objects of the present disclosure will become more apparent with reference to the following description taken in conjunction with the accompanying drawings wherein like reference numerals denote like elements and in which:

FIG. 1 illustrates an implantable medical device system in accordance with one or more embodiment of the present disclosure implanted in a human body.

FIG. 2 is a block diagram illustrating the various components of one or more embodiments of an implantable medical device configured to operate in accordance with the present disclosure.

FIG. 3 is a perspective view illustrating various relational positions of a patient in accordance with one or more embodiments of the present disclosure.

FIG. 4 is an operational flow diagram illustrating a process for optimizing or compensating for the implanted orientation of an implantable medical device in a patient in accordance with one or more embodiments of the present disclosure.

FIG. 5 is a graphical illustration of an example accelerometer signal measured in an implantable medical device while a patient is walking in accordance with one or more embodiment of the present disclosure.

FIG. 6 is a perspective block diagram illustrating the movement of an external magnet with respect to a patient in accordance with one or more embodiments of the present disclosure.

### DETAILED DESCRIPTION

A method and device are provided for detecting the implanted orientation of an implantable medical device in a patient and then optimizing or compensating for such orientation. A simplified optimization or compensation of the implanted orientation of an implantable medical device within the patient is provided in order to allow accelerometer signals to be adjusted to compensate for the implanted orientation so that they can be used to accurately detect physical changes in the physical activity of the patient in three orthogonal directions. In the following description, numerous embodiments are set forth in order to provide a thorough understanding of the invention. It will be apparent, however, to one skilled in the art, that these and other embodiments may be practiced without these specific details. In some instances, features well-known to those skilled in the art have not been described in detail in order not to obscure the present disclosure.

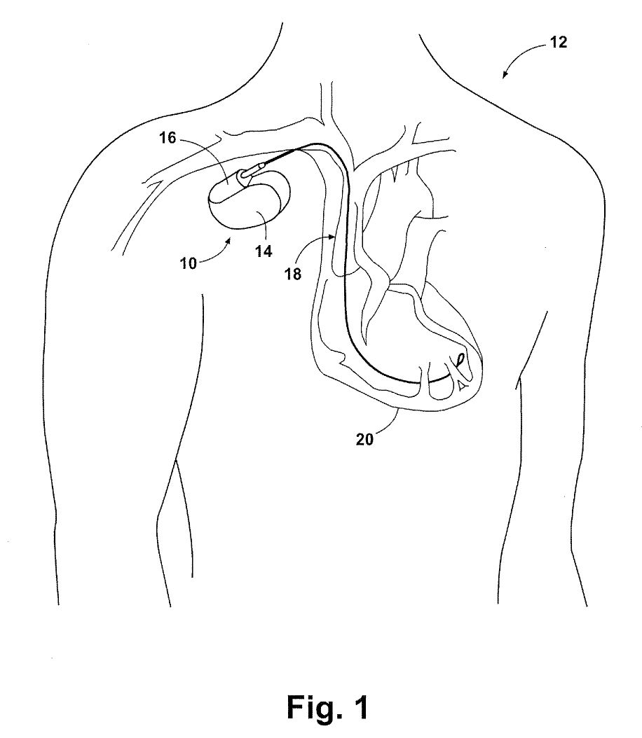

FIG. 1 is a simplified schematic view of one embodiment of implantable medical device (“IMD”) 10 of the present disclosure implanted within a human body 12. IMD 10 comprises a hermetically sealed enclosure 14 and connector module 16 for coupling IMD 10 to electrical leads 18 arranged within body 12, such as pacing and sensing leads 18 connected to portions of a heart 20 for delivery of pacing pulses to a patient's heart 20 and sensing of heart 20 conditions. While IMD 10 is depicted in a pacemaker device configuration in FIG. 1, it is understood that IMD 10 may comprise any type of subcutaneous or implanted device including, but not limited to implantable cardioverter-defibrillators (ICDs), an implantable combination pacemaker-cardioverter-defibrillator (PCDs), implantable brain stimulators, implantable gastric system stimulators, implantable nerve stimulators or muscle stimulators, implantable lower colon stimulators, implantable drug or beneficial agent dispensers or pumps, implantable cardiac signal loops or other types of recorders or monitors, implantable gene therapy delivery devices, implantable incontinence prevention or monitoring devices, implantable insulin pumps or monitoring devices, and so on.

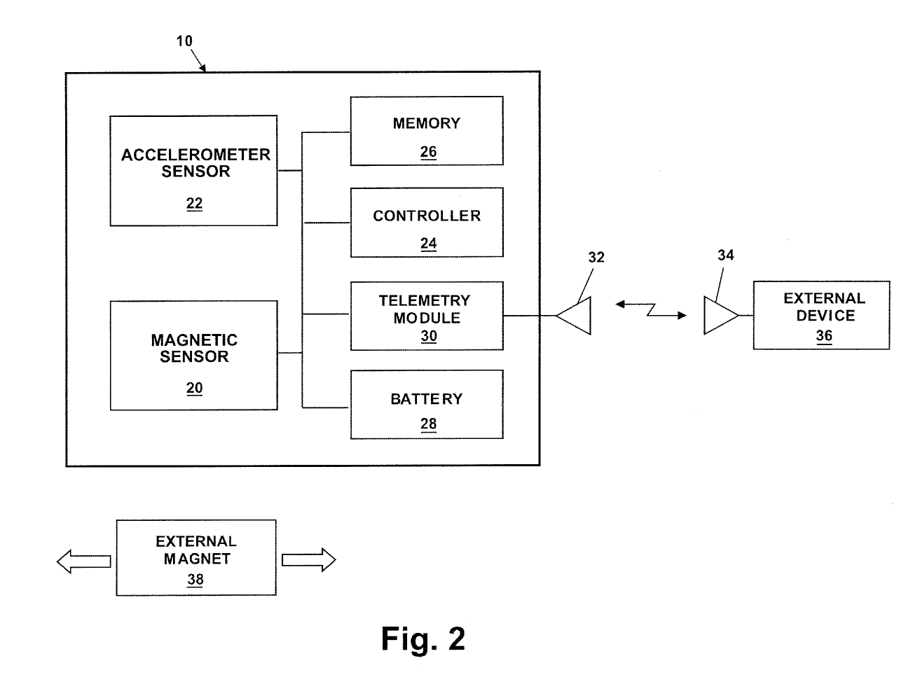

FIG. 2 is a block diagram illustrating the constituent components of IMD 10 in accordance with one embodiment having a microprocessor-based architecture. IMD 10 is shown as including magnetic sensor 20, accelerometer sensor 22, processor or controller 24, memory 26, battery 28, telemetry module 30, and other components as appropriate to produce the desired functionalities of the device.

IMD 10 may further include additional sensors configured to sense at least one physiological signal or condition, from which a physiological parameter can be determined. Such sensors can monitor electrical, mechanical, chemical, or optical information that contains physiological data of the patient and can utilize any source of physiological signals used for physiological events or conditions. For example, IMD 10 may include a heart sensor, such as the MDT Reveal® system, commercially available from Medtronic of Minneapolis, that is capable of sensing cardiac activity, electrocardiograms, heart rate, or the like. Reveal is a registered trademark of Medtronic, Inc. of Minneapolis, Minn.

Telemetry module 30 may comprise any unit capable of facilitating wireless data transfer between IMD 10 and an external device 36, where external device 36 may comprise an external medical device, a programming device, a remote telemetry station, a physician-activated device, a patient-activated device, a display device or any other type of device capable of sending and receiving signals to and from IMD 10. In one or more embodiments, external device 36 may be included within a patient activator device or a portable device wearable or capable of being carried by the patient. In one or more embodiments, external device 36 may comprise an in-home monitoring device, such as the Medtronic CareLink® Network monitor, that collects information from IMDs implanted in patients and communicates such information to remote clinicians through the Internet, phone lines or wireless networks. Carelink is a registered trademark of Medtronic, Inc. of Minneapolis, Minn. In one or more embodiments, external device 36 may comprise a personal computer or mobile phone having a software program installed thereon configured for receiving data from IMD 10, processing such data and/or further communicating such data to a remote location or clinician for further analysis and/or processing.

Telemetry module 30 and external device 36 are respectively coupled to antennas 32 and 34 for facilitating the wireless data transfer. Telemetry module 30 may be configured to perform any type of wireless communication. For example, telemetry module 30 may send and receive radio frequency (RF) signals, infrared (IR) frequency signals, or other electromagnetic signals. Any of a variety of modulation techniques may be used to modulate data on a respective electromagnetic carrier wave. Alternatively, telemetry module 30 may use sound waves for communicating data, or may use the patient's tissue as the transmission medium for communicating with a programmer positioned on the patients skin. In any event, telemetry module 30 facilitates wireless data transfer between IMD 10 and external device 36.

Controller 24 may comprise any of a wide variety of hardware or software configurations capable of executing algorithms to utilize data received from magnetic sensor 20 or accelerometer sensor 22 to compute the implanted orientation of IMD 10. Example hardware implementations of controller 24 include implementations within an application specific integrated circuit (ASIC), a field programmable gate array (FPGA), a programmable logic device, specifically designed hardware components, one or more processors, or any combination thereof. If implemented in software, a computer readable medium, such as a memory in the IMD 10, may store computer readable instructions, e.g., program code, that can be executed by controller 24 to carry out one of more of the techniques described herein. For example, the memory may comprise random access memory (RAM), read-only memory (ROM), non-volatile random access memory (NVRAM), electrically erasable programmable read-only memory (EEPROM), flash memory, or the like.

In one or more embodiments, magnetic sensor 20 may include a three dimensional (“3D”) magnetic sensor. In one or more embodiments, magnetic sensor 20 may comprise a Hall sensor as are well known to those skilled in the art for measuring magnetic fields. It is understood that any magnetic sensor capable of measuring magnetic fields can be utilized as magnetic sensor 20. Magnetic sensor 20 is further arranged such that it is capable of sensing a magnetic field exerted on it from a moving magnetic field source positioned adjacent to the patient, such as external magnet 38 or another magnetic field source.

In one or more embodiments, accelerometer sensor 22 may include a 3D accelerometer sensor. U.S. Pat. No. 6,044,297 describes one example of a 3D accelerometer sensor that may be used as accelerometer sensor 22, and is incorporated herein by reference in its entirety. It is understood that other types of 3D accelerometers capable of measuring movement and/or orientation is multiple different directions can be utilized as accelerometer sensor 22.

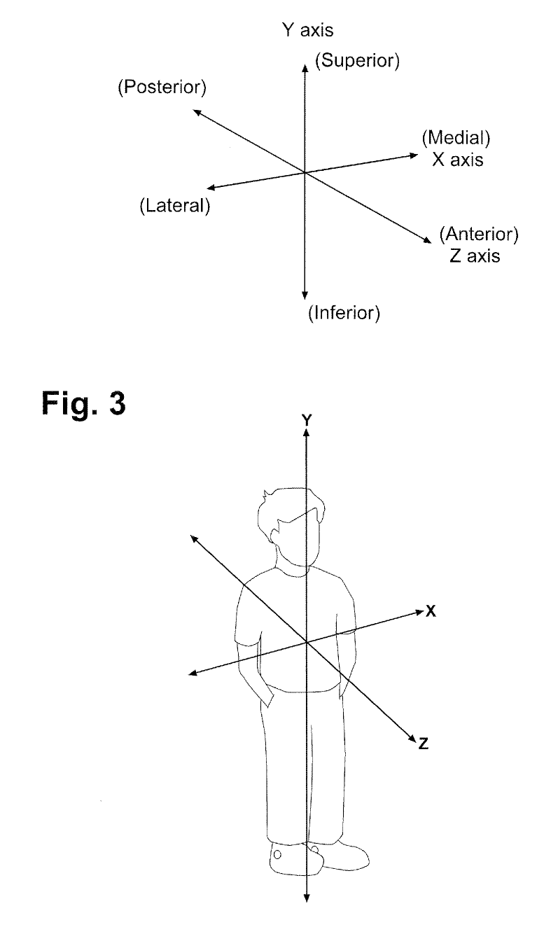

Referring now to FIG. 3, a perspective view of a patient in an upright position is illustrated showing the three orthogonal axes of a patient in accordance with one or more embodiments, namely the x-axis, y-axis and z-axis. The x-axis will also be referred to as the medial-lateral axis that extends along a direction from a medial side (e.g., left side) of the patent to a lateral side (e.g. right side) of the patient. The y-axis will also be referred to as the superior-inferior axis that extends along a direction from a superior side (e.g., top) of the patent to an inferior side (e.g., bottom) of the patient. The z-axis will also be referred to as the anterior-posterior axis that extends along a direction from an anterior side (e.g., front) of the patent to a posterior side (e.g., back) of the patient. The three orthogonal axis of the patient may not coincide exactly with the three orthogonal axes of the implanted IMD 10, since IMD 10 will generally possess an implanted orientation that must account for the space available within the patient where IMD 10 may be situated. Thus, the method and device are provided for optimizing or compensating for the implanted orientation of an implantable medical device within the patient, so that accelerometer signals received from accelerometer sensor 22 can be adjusted accordingly to provide a more accurate determination of the orientation and activity of the patient.

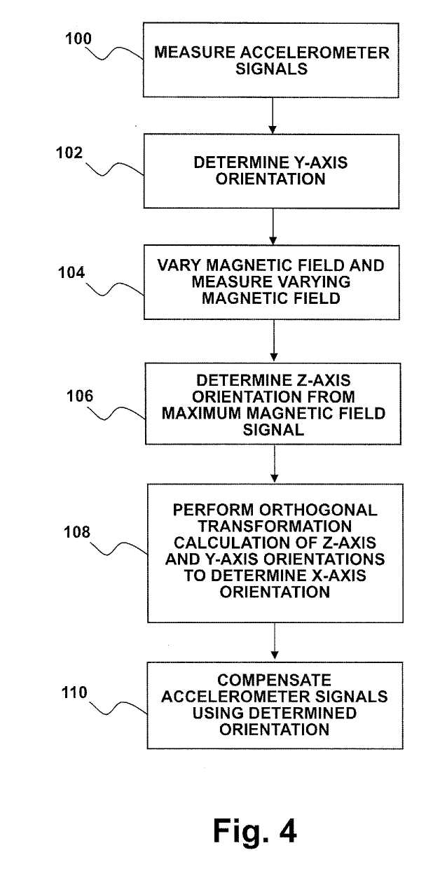

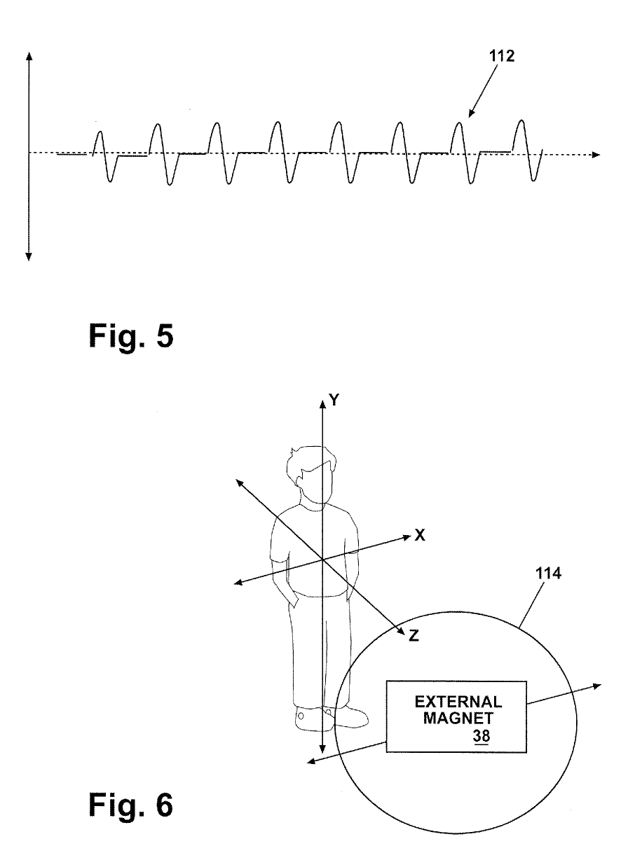

Referring now to FIG. 4, an operation flow diagram is provided for one or more embodiments of a method of optimizing or compensating for the implanted orientation of an implantable medical device within the patient. The accelerometer sensor 22 is preferably a 3D accelerometer configured for measuring acceleration signals in three orthogonal directional axes, namely x-axis, z-axis and y-axis. In step 100, accelerometer sensor 22 measures acceleration signals which are forwarded to controller 24 connected to accelerometer sensor 22 for determining a y-axis orientation of accelerometer sensor 22 or IMD 10 from the measured accelerometer signals in step 102. In some embodiments, the y-axis orientation of IMD 10 is determined when it is known or determined that the patient in which IMD 10 is implanted is in an upright position or posture. Since a patient will be in a substantially upright position while walking, in some embodiments, controller 24 may configured to sense when the patient is walking, jogging, running or performing another type of physical activity giving indication that the patient is in an upright position. For example, controller 24 may monitor the accelerometer signals received from accelerometer sensor 22 to detect the presence of a certain low frequency periodic signal that can be used to identify that the patient is walking, as shown by the periodic accelerometer signal 112 illustrated in FIG. 5. Once it is known or determined that the patient is in an upright or walking position, the y-axis orientation of accelerometer sensor 22 or IMD 10 may be determined from the gravitational force exerted on the accelerometer sensor 22, where such y-axis orientation calculations using gravitation force are well-known to those skilled in the art of accelerometers.

In one or more embodiments, a magnetic field is then applied to IMD 10 for use in determining the z-axis orientation of IMD 10. The magnetic field may be exerted on IMD 10 in any number of possible manners. In some embodiments, a magnetic field source (i.e., external magnet 38) having a stationary and known position may be positioned with respect to IMD 10 such that the a magnetic field will be imparted on magnetic sensor 22. The strength of the magnetic field exerted on magnetic sensor 22 from the stationary magnetic field source 38 can be measured and used with the known positional relationship of the magnetic field source 38 with respect to IMD 10 to calculate the z-axis orientation of the device. In one or more embodiments, a varying magnetic field may be utilized to calculate the z-axis orientation of the device in which an external magnet 38 may be moved on the anterior side of the patient along a back-and-forth direction substantially along a direction of the medial-lateral axis. For example, as illustrated in FIG. 6, the external magnet 38 is moved from one side (e.g., the left side) of the patient to the other side (e.g., the right side) of the patient on the front side of the patient. As the external magnet 38 moves, the magnetic field 114 exerted on IMD 10 and magnetic sensor 22 will vary. Magnetic sensor 22 senses and measures the varying strength of the magnetic field exerted on it by the magnetic field 114 in step 104, where the sensed magnetic field signals are communicated to controller 24 for further analysis. In one or more embodiments, controller 24 monitors the values of the received magnetic field signals and determines when the maximum value of the magnetic field exerted on magnetic sensor 22 occurs. Controller 24 then identifies in step 106 the corresponding location of external magnet 38 associated with when the maximum magnetic field value occurs. In one or more embodiments, the z-axis orientation of IMD 10 is determined based on the location at which the magnetic field has a maximum value. In other words, the z-axis (anterior-posterior axis) will extend through IMD 10 and the location of external magnet 38 at which the magnetic field has a maximum value.

In one or more embodiments, the varying magnetic field between magnetic sensor 22 and external magnet 38 can likewise be achieved by retaining external magnet 38 in a stationary position while the magnetic sensor 22 (i.e., the patient) moves with respect to external magnet 38. In such stationary relationships of the external magnet 38, external magnet 38 may be situated within external device 36. In some embodiments, external magnet 38 may be situated within other devices or components in at least one of the patient's home, the physician's office, a hospital or a clinician. In one or more embodiments, a magnetic field source other than an external magnet 38 may be utilized to generate the varying magnetic field.

In one or more embodiments, the orientation between the accelerometer sensor 22 and magnetic sensor 20 within IMD 10 is known and fixed. Due to this known relationship, controller 24 may be configured for determining an x-axis orientation of IMD 10 from an orthogonal transformation calculation using the previously determined y-axis orientation obtained from the accelerometer signals and the z-axis orientation from the magnetic field signals. In this manner, once the x-axis, y-axis and z-axis orientations of IMD 10 are known, the implanted 3D orientation of IMD 10 with respect to the patient can be determined. Once the implanted orientation is known, the accelerometer signals received from accelerometer sensor 22 can be compensated, adjusted or corrected either by controller 24, accelerometer sensor 22 itself or another device to account for the implanted orientation. In this manner, accelerometer sensor 22 can be calibrated such that its output signals can be adjusted to account for the implanted orientation of the implantable medical device in the patient to provide a more accurate representation of the patient's activity and orientation. In some embodiments, the calibration procedures can be repeated at various points in time to re-calibrate or compensate for the implanted orientation of IMD to account for movement of IMD 10 within the patient over time.

While the various embodiments herein have been described with respect to an implantable or subcutaneous device in which the orientation of the medical device may not be easily adjustable, the invention may also be implemented in external medical devices for providing a quick, automated manner of determining orientation of the medical device with respect to the patient.

In one or more embodiments, the calculated calibration or compensation procedures can be performed using programs operating on IMD 10, on an in-home patient monitoring system 36, or in the physician's office. The calculated calibration or compensation values may then be stored in memory 26 of IMD 10 and used by controller 24 in compensating future accelerometer signals received from accelerometer sensor 22.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method of determining the orientation of an implantable medical device, comprising:
determining a y-axis orientation of the device using accelerometer signals from a 3D accelerometer positioned within the device using gravity as an external force;
determining an z-axis orientation of the device based on properties of a magnetic field between a magnetic sensor positioned within the device and an external magnet; and
determining an x-axis orientation of the device from an orthogonal transformation calculation using the previously determined y-axis and z-axis orientations.

2. The method of claim 1, further comprising determining the y-axis orientation when a patient in which the implantable medical device is implanted is in an upright posture.

3. The method of claim 1, further comprising:
monitoring the accelerometer signals from the 3D accelerometer to detect certain low frequency periodic signals signifying that a patient in which the implantable medical device is implanted is involved in an upright activity.

4. The method of claim 1, further comprising determining the z-axis orientation by:
moving the external magnet and magnetic sensor with respect to each other between so that the external magnet moves between lateral and medial positions of a patient in which the implantable medical device is implanted;
measuring a strength of a magnetic field imparted on the magnetic sensor by the moving external magnet;
identifying the location at which the magnetic field between the external magnet and magnetic sensor has a maximum value; and
determining the z-axis orientation of the device based on the location at which the magnetic field has a maximum value.

5. The method of claim 1, further comprising determining the z-axis orientation by:
positioning the external magnet such that the external magnet includes a stationary, known position with respect to the magnetic sensor;
measuring a strength of a magnetic field imparted on the magnetic sensor by the external magnet;
determining the z-axis orientation of the device based on the strength of the magnetic field imparted on the magnetic sensor and the known position of the external magnet.

6. The method of claim 1, further comprising calibrating the 3D accelerometer using the determined y-axis, z-axis and x-axis orientations.

7. The method of claim 1, further comprising positioning the external magnet in an in-home patient monitoring system so that a patient can compensate the orientation of the device by moving with respect to the in-home patient monitoring system to determine compensate the x-axis orientation.

8. An implantable medical device comprising:
a 3D accelerometer sensor configured for measuring acceleration signals in three orthogonal directional axes comprising an x-axis, z-axis and y-axis;
a magnetic sensor configured to sense a magnetic field exerted on the magnetic sensor from a moving magnetic field source; and
a controller coupled to the 3D accelerometer for receiving acceleration signals and to the magnetic sensor for receiving magnetic field value signals,
the controller configured for determining a y-axis orientation of the device from a y-axis acceleration signal received from the 3D accelerometer, the controller further configured for determining an z-axis orientation based on magnetic field value signals received from the magnetic sensor, the controller further configured for determining an x-axis orientation of the device from an orthogonal transformation calculation using the previously determined y-axis and z-axis orientations.

9. The implantable medical device of claim 8, wherein the controller is configured to determine the y-axis orientation of the device when a patient in which the device is implanted is positioned in an upright posture.

10. The implantable medical device of claim 8, wherein the controller is configured to monitor the accelerometer signals received from the 3D accelerometer sensor to detect certain low frequency periodic signals signifying that a patient in which the implantable medical device is implanted is involved in an upright activity, wherein the y-axis orientation of the device is determined when the patient is determined to be involved in the upright activity.

11. The implantable medical device of claim 8, wherein the controller is configured to determine the z-axis orientation by:
monitoring magnetic field value signals received from the magnetic sensor from a magnetic field imparted on the magnetic sensor from an external magnet that is moved with respect to the magnetic sensor between lateral and medial positions of a patient in which the device is implanted;
identifying a magnetic field value signal having a maximum value and identifying a corresponding position of the external magnet when generating the maximum magnetic field value signal; and
determining the z-axis orientation of the device based on the location at which the magnetic field value signal has a maximum value.

12. The implantable medical device of claim 8, wherein the controller is configured to calibrate the 3D accelerometer using the determined y-axis, z-axis and x-axis orientations.

13. The implantable medical device of claim 8, wherein the external magnet in positioned in an in-home patient monitoring system so that a patient can compensate the orientation of the device by moving with respect to the in-home patient monitoring system to determine compensate the x-axis orientation.

14. An implantable medical device, comprising:
means for determining a y-axis orientation of the device using accelerometer signals from a 3D accelerometer positioned within the device;
means for determining an z-axis orientation of the device based on properties of a magnetic field between a magnetic sensor positioned within the device and an external magnet; and
means for determining an x-axis orientation of the device from an orthogonal transformation calculation using the previously determined y-axis and z-axis orientations.

15. The implantable medical device of claim 14, wherein the means for determining the y-axis orientation is further configured for determining the y-axis orientation when a patient in which the implantable medical device is implanted is in an upright posture.

16. The implantable medical device of claim 14, wherein the means for determining the y-axis orientation is further configured for monitoring the accelerometer signals from the 3D accelerometer to detect certain low frequency periodic signals signifying that a patient in which the implantable medical device is implanted is involved in an upright activity.

17. The implantable medical device of claim 14, wherein the means for determining the z-axis orientation is further configured for:
monitoring magnetic field value signals received from the magnetic sensor from a magnetic field imparted on the magnetic sensor from an external magnet that is moved with respect to the magnetic sensor between lateral and medial positions of a patient in which the device is implanted;
identifying a magnetic field value signal having a maximum value and identifying a corresponding position of the external magnet when generating the maximum magnetic field value signal; and
determining the z-axis orientation of the device based on the location at which the magnetic field value signal has a maximum value.

18. The implantable medical device of claim 14, wherein the means for determining the z-axis orientation is further configured for:
monitoring magnetic field value signals received from the magnetic sensor from a magnetic field imparted on the magnetic sensor from an external magnet that is positioned in a stationary, known position with respect to the magnetic sensor;
measuring a strength of the magnetic field imparted on the magnetic sensor by the external magnet;
determining the z-axis orientation of the device based on the strength of the magnetic field imparted on the magnetic sensor and the known position of the external magnet.

19. The implantable medical device of claim 14, further comprising means for calibrating the 3D accelerometer using the determined y-axis, z-axis and x-axis orientations.

20. The implantable medical device of claim 14, further comprising positioning the external magnet in an in-home patient monitoring system so that a patient can compensate the orientation of the device by moving with respect to the in-home patient monitoring system to determine compensate the x-axis orientation.
